(19) United States
(12) Patent Application Publication
(10) Pub. No.: US 2011/0201585 A1
(43) Pub. Date: Aug. 18, 2011

(54) The Use of Oestrogen Applied to the Penis to Reduce HIV Infections

(76) Inventors: Roger Short, Victoria (AU); Andrew Pask, Victoria (AU)

(21) Appl. No.: 12/599,753

(22) PCT Filed: May 9, 2008
(86) PCT No.: PCT/AU2008/000650

§ 371 Date: Nov. 11, 2009

(30) Foreign Application Priority Data
May 11, 2007 (AU) 2007902523

(51) Int. Cl. A61K 31/565; A61K 31/566; C07J 1/00; A61P 31/18; A61F 6/04

(52) U.S. Cl. 514/178; 514/182; 552/617; 552/625; 128/844

(57) ABSTRACT

A method of reducing or inhibiting the risk of infection from sexually transmitted viral infections (STIs), such as HIV and HPV, comprising the topical administration of oestrogenic compounds, analogues or metabolites thereof or oestrogen agonists to a human penis. A method to increase keratinisation of the penile epithelium is also provided. Suitable formulations are also provided.

### FIELD OF THE INVENTION

The present invention relates to a method of reducing the transmission of sexually transmitted infections such as HIV and HPV by minimising contact of the infective agent with Langerhans cells of the foreskin.

### BACKGROUND OF THE INVENTION

The control, prevention and management of sexually transmitted infections (STIs), particularly those'caused by viral agents, represents a significant healthcare challenge world wide. STIs are a major global cause of acute illness, infertility and death for millions of men, women and infants. Sexually transmitted infections include Human Immunodeficiency Viruses (HIV-1 and HIV-2) and Human Papillomavirus (HPV). In 2006, about 40 million people were currently infected with HIV-1, with 4.3 million new infections and 3 million deaths. Other STIs, such as certain strains of human papillomavirus (HPV) (including types 16, 18, 31 and 45) can lead to cancer including cervical and penile cancer.

In the case of HIV, over 80% of infections occur during heterosexual intercourse (Short, 2006). This is despite the fact that the probability of transmission per act of sexual intercourse may be as low as 1 in 1000 (Royce et al. 1997). Education programs and the use of condoms have certainly been valuable in combating the epidemic, particularly in Western cultures. However, the spread of infection continues unabated in many developing countries, and a need exists for new ways to prevent infection from occurring in the first place.

Despite the high incidence of heterosexual transmission of HIV, very little is known about the route of HIV entry into the male reproductive tract. Male circumcision has been demonstrated to have a protective effect, providing men with at least 60% reduced risk infection (Newell & Barninghausen 2007). This protection is thought to be due to the removal of most of the inner foreskin, which has been demonstrated to be the principal site of HIV entry into the penis (McCoombe & Short, 2006), as illustrated in FIG. 1. A diagrammatic section of a human penis in FIG. 1 shows that in a flaccid penis, FIG. 1(a), the foreskin 1 covers the glans penis 5, inner foreskin 3 is covered by the outer foreskin 2. The urethral meatus 4 and frenulum 8 are also shown in FIG. 1(a). In an erect penis in FIG. 1(b) where the foreskin is retracted, the inner foreskin 3 is exposed. This exposure of the inner foreskin 3 increases the number of potential sites of HIV entry 7. Other areas of an erect penis are sites 6 of no or minimal HIV entry.

The inner foreskin is rich in Langerhans cells. Langerhans cells (LCs) are an immature subset of Dendritic cells (DCs) present in the epithelium. The dendritic processes of the LCs lie just beneath the surface of the epithelium, and contain specific viral receptors, such as HIV-specific receptors. After binding to these receptors, HIV is internalised, where a C-type lectin, langerin, binds to and degrades the virus, thereby preventing the transfer of the virus to T cells (de Witte et al. 2007; Schwartz 2007). However, high viral loads deplete the langerin, resulting in the LCs becoming vectors for transporting the virus to regional lymph nodes, where it can then infect naïve T cells and establish a systemic infection.

As noted above, protection by circumcision is only about 60%, so even after circumcision it is still recommended that condoms be used to prevent infection (Newell and Baminghausen 2007). In addition, circumcision is unacceptable in some cultures e.g. Hindus, and simply not recommended in others e.g. Buddhists.

Reference to any prior art in the specification is not, and should not be taken as, an acknowledgment, or any form of suggestion, that this prior art forms part of the common general knowledge in Australia or any other jurisdiction or that this prior art could reasonably be expected to be ascertained, understood and regarded as relevant by a person skilled in the art.

### SUMMARY OF THE INVENTION

In one aspect of the invention, there is provided a method of reducing or inhibiting the risk of infection from sexually transmitted viral infections (STIs), such as HIV and HPV, comprising the topical administration of oestrogenic compounds, analogues or metabolites thereof or oestrogen agonists to a human penis. Without being bound by any theory or mode of action, the inventors believe that the inner foreskin responds to topical oestrogen administration by hypertrophy and increased keratinisation. This should protect the dendritic processes of the LCs from coming into contact with the virus, and significantly reduce the viral load of HIV or HPV entering the body.

Unexpectedly, the current inventors have identified the presence of oestrogen receptors in the human foreskin in high concentration. Topically applied oestrogen has been shown to induce thickening and keratinisation of the vaginal epithelium, and studies conducted in monkeys have shown that this provides significant protection against simian immunodeficiency virus infection (Smith et al. 2004). Without being bound by any theory or mode of action, the inventors believe that the foreskin is the endocrine homologue of the vagina, rapidly responding to topical oestrogen administration by thickening and keratinisation.

Therefore, the inventors have sought to enhance the effectiveness of what is believed to be a natural defence mechanism of thickening and keratinising the epithelium of the inner aspect of the foreskin by oestrogen, or other compounds that induce keratinisation, to reduce viral contact with the LCs. This provides a reversible medical alternative to surgical circumcision for reducing or inhibiting the risk of infection from sexually transmitted infections (STIs), such as HIV and HPV.

Accordingly, the invention provides a method of reducing or inhibiting the risk of infection from sexually transmitted viral infections (STIs), such as HIV and HPV, by increasing keratinisation of the human penile epithelium of a penis, especially the inner foreskin of the penis. This may be achieved by topical administration of oestrogenic compounds, analogues or metabolites thereof, or oestrogen agonists to the penis.

In another embodiment, there is provided a method of increasing keratinisation of the human penile epithelium of a penis, especially the inner foreskin of the penis, by topical administration of oestrogenic compounds, analogues or metabolites thereof, or oestrogen agonists to the penis. Normally, the inner foreskin of the penis has a relatively thin overlying layer of keratin. The method is also suitable for use by circumcised males, to induce keratinisation of any inner foreskin remnants not completely removed by circumcision. Alternatively, the method is suitable for use by uncircumcised males as a ‘stop gap’ treatment while they await circumcision.

There is also provided a method of decreasing the exposure of Langerhans cells to sexually transmitted infectious agents by topical administration of oestrogenic compounds, analogues or metabolites thereof or oestrogen agonists to a penis. This in turn reduces the risk of or inhibits infection from sexually transmitted infections (STIs), such as HIV and HPV.

In an alternative embodiment, the methods of the invention may also comprise the topical administration of non-oestrogenic compounds that induce keratinisation.

In another aspect of the invention, there is provided a topical oestrogenic compound, analogues or metabolites thereof or oestrogen agonist formulation suitable for use in the methods of the invention to reduce or inhibit the risk of infection from sexually transmitted infections (STIs), such as HIV and HPV, to thicken and/or keratinise the epithelium of the penis, and to decrease the exposure of Langerhans cells to STIs. Preferably the oestrogenic compound is a relatively weak natural oestrogen, such as oestriol to avoid systemic side effects such as gynaecomastia, pituitary inhibition or infertility. Furthermore, this will reduce the risk of environmental contamination. Alternatively, substances such as xenooestrogens (i.e. oestrogen analogues and synthetic oestrogens) and phytooestrogens that possess oestrogenic activity are also suitable for use in the formulations of the invention.

In one embodiment, the formulations are adapted for external topical application. Suitable forms include thin formulations, e.g. aqueous compositions, for spraying or applying for ready absorption.

The oestrogenic compounds, analogues or metabolites thereof and oestrogen agonists of the formulations, or the non-oestrogenic formulations comprising compounds that induce keratinisation, may be applied in any therapeutically effective amount. Oestriol is a suitable oestrogenic compound. A therapeutically effective amount means the minimum amount to achieve efficacy for the methods of the invention. Such amounts will of course depend on, among other things, individual patient parameters and the STI. Methods for determining a suitable amount are well known to those of ordinary skill in the art and can be addressed with no more than routine experimentation. In one embodiment, a cream having 10 μg-1 mg oestriol is applied topically once daily for 10-20 days. In one form, about 0.5 ml of a cream having 1 mg/ml oestriol is used. Preferably, this administration is followed by one weekly administration of the same amount. A reduced dose more often to deliver the same amount is also included. In one embodiment, each dose is between 5 μg-1 mg.

The formulations of the invention are also useful for reducing risk of HIV infection in discordant homosexual couples. Mutual masturbation without condoms for example may result in the infected ejaculate from one partner coming in contact with the penis of the other partner, thereby exposing the inner foreskin to STIs in the ejaculate. Alternatively, the infected ejaculate may be used as a lubricant, and is intentionally rubbed over the entire shaft and head of the penis. This again may expose the inner foreskin to STIs present in the ejaculate of an infected partner.

In yet another embodiment, the formulations of the invention are also useful as a short-term pre- and post-operative medication for those about to undergo surgical circumcision. Again, without being bound by any theory or mode of action, it is believed that the mitogenic activity of oestrogen assists in post-operative wound healing, and prevents the increased risk of infection before the circumcision wounds have healed.

Formulations of the present invention also include, but are not limited to, antibiotics suitable for skin treatment such as chloramphenicol, chlortetracycline, clyndamycin, clioquinol, erythromycin, framycetin, gramicidin, fusidic acid, gentamicin, mafenide, mupiroicin, neomycin, polymyxin B, bacitracin, silver sulfadiazine, tetracycline and chlortetracycline.

In yet another embodiment, there is provided a use of oestrogenic compounds, analogues or metabolites thereof or oestrogen agonists in the manufacture of a medicament for reducing or inhibiting the risk of infection from sexually transmitted viral infections (STIs), such as HIV and HPV.

In yet another embodiment, there is provided a use of oestrogenic compounds, analogues or metabolites thereof or oestrogen agonists in the manufacture of a medicament for increasing keratinisation of a human penile epithelium of a penis, especially the inner foreskin of the penis.

In yet another embodiment, there is provided a use of oestrogenic compounds, analogues or metabolites thereof or oestrogen agonists in the manufacture of a medicament for decreasing the exposure of Langerhans cells in a human penis to sexually transmitted infectious agents.

In yet another embodiment, there is provided a use of compounds that induce keratinisation in the manufacture of a medicament for reducing or inhibiting the risk of infection from sexually transmitted viral infections (STIs), such as HIV and HPV.

In yet another embodiment, there is provided a use of compounds that induce keratinisation in the manufacture of a medicament for increasing keratinisation of a human penile epithelium of a penis, especially the inner foreskin of the penis.

In yet another embodiment, there is provided a use of compounds that induce keratinisation in the manufacture of a medicament for decreasing the exposure of Langerhans cells in a human penis to sexually transmitted infectious agents.

In the above three embodiments, in one form the compounds are oestrogenic. Oestriol is a suitable compound. In another form, the compounds are non-oestrogenic.

In yet another embodiment, there is provided oestrogenic compounds, analogues or metabolites thereof or oestrogen agonists for use in reducing or inhibiting the risk of infection from sexually transmitted viral infections (STIs), such as HIV and HPV.

In yet another embodiment, there is provided oestrogenic compounds, analogues or metabolites thereof or oestrogen agonists for use in increasing keratinisation of a human penile epithelium of a penis, especially the inner foreskin of the penis.

In yet another embodiment, there is provided oestrogenic compounds, analogues or metabolites thereof or oestrogen agonists for use in decreasing the exposure of Langerhans cells in a human penis to sexually transmitted infectious agents.

In yet another embodiment, there is provided a formulation for reducing or inhibiting the risk of infection from sexually transmitted viral infections (STIs), such as HIV and HPV having as an active ingredient oestrogenic compounds, analogues or metabolites thereof or oestrogen agonists.

In yet another embodiment, there is provided a formulation for increasing keratinisation of a human penile epithelium of a penis, especially the inner foreskin of the penis having as an active ingredient oestrogenic compounds, analogues or metabolites thereof or oestrogen agonists.

In yet another embodiment, there is provided a formulation for decreasing the exposure of Langerhans cells in a human penis to sexually transmitted infectious agents having as an active ingredient oestrogenic compounds, analogues or metabolites thereof or oestrogen agonists.

In addition to providing a reversible medical alternative to surgical circumcision as a way of reducing or inhibiting STIs such as HIV and HPV from establishing infection, the formulations may be administered as a condom lubricant. In one embodiment of the invention, there is provided a formulation of the invention that is compatible with condoms. In another embodiment, there is provided a condom having a coating on at least part of its surface containing a formulation according to the invention.

The formulation preferably covers the entire inner and outer surface of the condom. Regular contact of a penis with the formulation will lead over time to an increase in keratin and therefore decrease the risk of HIV and HPV infection. Similarly, in females, regular contact over time will lead to a thickening of the vaginal keratin and decrease risk of HIV and HPV infection.

Suitable formulations for topical administration include soluble tablets, solutions, suspensions, gels, oils, lotions, sprays, transdermal patches, foams, films, jellies, and creams as well as discrete units such as microencapsulated suspensions. Other delivery systems include sustained release delivery systems which can provide for slow release of the active component of the invention, including sustained release gels, soluble tablets or creams.

The formulations of the present invention preferably include a carrier, excipient or diluent. The carrier, excipient or diluent may include one or more of any and all conventional solvents, dispersion media, fillers, solid carriers, aqueous solutions, coatings, viscosity modifying agents, antimicrobial agents, isotonic, and absorption enhancing or delaying agents, activity enhancing or delaying agents and the like. The use of such media and agents is well known in the art, and it is described, by way of example, in Remington's Pharmaceutical Sciences, 18th Edition Mack Publishing Company, Pennsylvania, USA 1990. Except insofar as any conventional carrier and/or diluent is incompatible with the active ingredient, use thereof in the formulations of the present invention is contemplated.

It will be appreciated that formulations of the invention intended to be carried on a condom, or used as an adjunct to condom use, will be formulated such that the ingredients (alone or in combination) will not compromise condom integrity.

Suitable compositions to act as lubricants in this embodiment include the above formulations of the present invention and also silicone based condom lubricants or components of silicone based condom lubricants. Components of typical condom lubricants include water, dimethicone, hexamethyl disiloxane, docosamethly decasiloxane, tocopheryl acetate, carbomer, sodium hydroxide, propylene glycol, benzyl alcohol, methylchloroisothiazolinone, methylisothiazolinone, cyclomethicone and dimethiconol. For example, known combinations suitable for combination with a formulation according to the invention are dimethicone, hexamethyl disiloxane, docosamethly decasiloxane, and tocopheryl acetate; another is aqua, dimethicone, carbomer, sodium hydroxide, propylene glycol, benzyl alcohol, methylchloroisothiazolinone and methylisothiazolinonel; another is cyclomethicone, dimethicone and dimethiconol.

As used herein in this specification and claims, the singular forms “a”, “an” and “the” include plural referents unless the context clearly dictates otherwise.

It will also be understood that the term “comprises” (or its grammatical variants) as used in this specification is equivalent to the term “includes” and should not be taken as excluding the presence of other elements or features.

### BRIEF DESCRIPTION OF THE FIGURES

FIG. 1 is a cross section illustration of a penis and representation of sites of HIV entry or sites of no or minimal HIV entry.

FIG. 2 is a histological analyses of the inner and outer aspects of the foreskin of the penis, stained for the presence and localisation of oestrogen receptors.

FIG. 3 is a histological analysis of the inner foreskin demonstrating that topical oestrogen treatment leads to an increase in the thickness of the inner foreskin epithelium and overlying keratin.

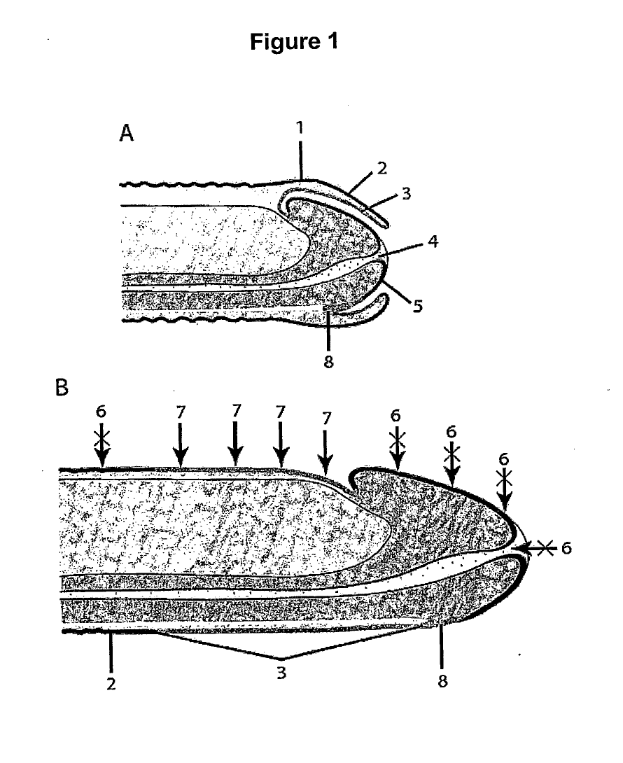

### EXAMPLES

It will be understood that the invention disclosed and defined in this specification extends to all alternative combinations of two or more of the individual features mentioned or evident from the text or drawings. All of these different combinations constitute various alternative aspects of the invention.

### Example 1

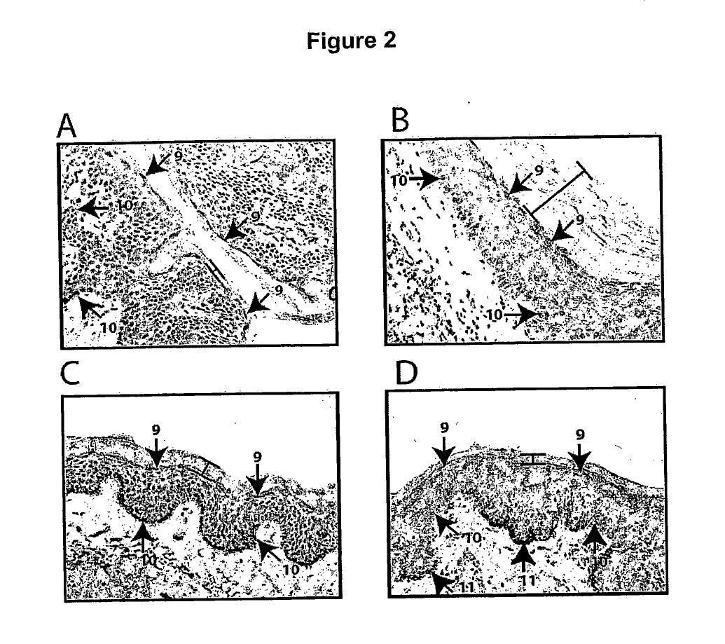

### Localisation of Oestrogen Receptors (FIG. 2)

Sections of the inner (FIG. 2; panels A, C, F and G) and outer foreskin (FIG. 2; panels B, D, E and H) were prepared and stained by immunohistochemical staining to determine the presence and distribution of ERα (panels A and B) and ERβ (panels C, D, E and F) protein (FIG. 2). Immunohistochemical staining refers to the process of detecting molecules in cells of a tissue section taking advantage of the principle of antibodies binding specifically to antigens. Antibodies to ERα or ERβ were added to the tissue sections followed then by an antibody to detect the ERα and ERβ antibodies which then allows visualisation.

The presence of either ERα or ERβ is shown by dark staining on panels A to F. ERα staining was strongest in the stratum granulosum cells 9 and in the stratum basale 10 with weakest staining in the stratum spinosum. Panels A, B, C, D, G and H are at the same magnification, so the natural variation in the thickness of the keratin layer is evident between samples as shown by the black distance bars. ERβ shows a similar localisation to ERα, but was more prevalent in the dermis than ERα. Panels E and F are magnified views of panel D and C respectively. These panels show characteristic cytoplasmic, nuclear and peri-nuclear staining for ERα and ERβ in the epithelium. Negative control samples of the inner (panel G) and outer foreskin (panel H) were not incubated with an antibody to either ERα and ERβ but instead with an equivalent amount of non-specific rabbit Immunoglobulin G. As expected there was no staining observed in the negative control samples suggesting that the staining in panels A to F was specific for ERα and ERβ protein. Melanocytes are present in some sections as dark cells found at the base of the epidermis 11 and do not indicate staining.

### Example 2

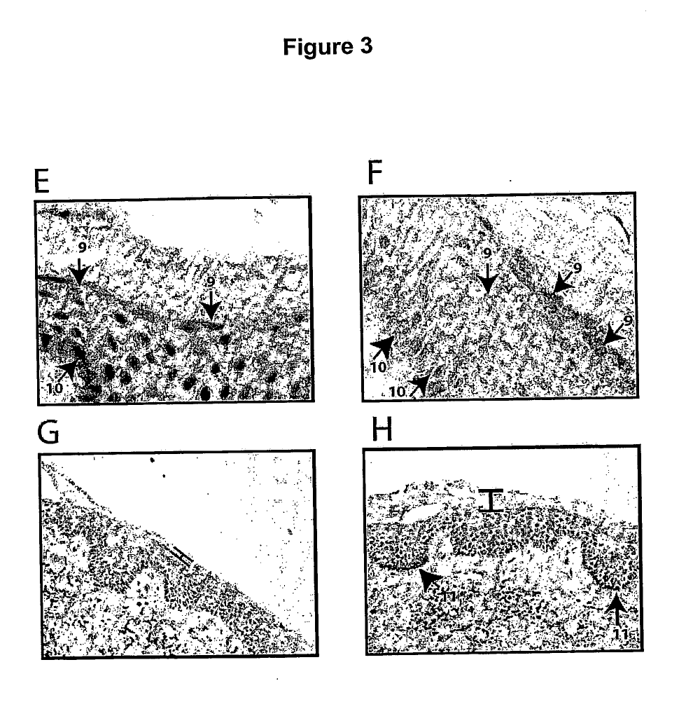

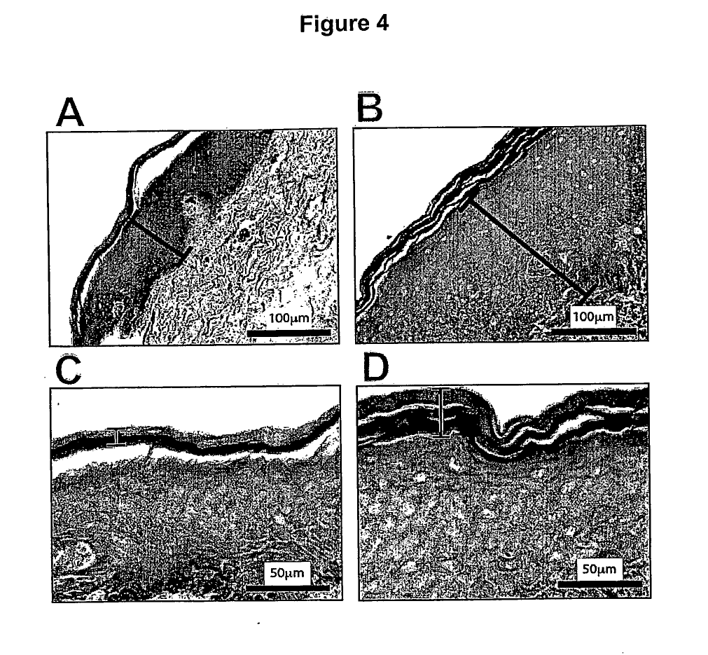

### Responsiveness of the Foreskin to Topical Oestrogen Administration (FIG. 3)

Examination of the Inner foreskin was conducted by routine histology methods before and after topical oestrogen treatment to examine whether topical oestrogen treatment leads to an increase in the thickness of the inner foreskin epithelium and overlying keratin layer. The epithelial histology from a pre-treatment inner foreskin biopsy, collected prior to topical oestrogen administration, was used to determine the baseline epithelium thickness and keratin thickness for the individual. Panels A and C of FIG. 3 show the structure of an inner foreskin epithelium biopsy sampled prior to oestrogen treatment. This sample was compared with the histology after treatment with topical oestrogen (panels B and D).

The pre-treatment biopsy wound was allowed to heal for two weeks prior to application of the topical oestrogen cream. The individual then applied 0.5 ml of a topical oestriol cream (Ovestin-Registered Trade Mark of Organon, 1 mg/ml oestriol), to at least the inner aspect of the foreskin, daily for 14 days. At the end of the treatment period the foreskin was collected. Panels B and D of FIG. 3 show the structure of the inner foreskin following daily application of the topical oestriol cream for 14 days. The thickness of the epithelium is indicated by black bars in panels A and B. The inner foreskin epithelium after topical oestriol treatment (panel B) has increased to over twice the thickness that it was before treatment (panel A) and there is also a concomitant increase in the thickness of the overlying keratin (indicated by black bars in panel C and D) of the inner foreskin. The post-treatment section (panel D) shows an overlying keratin being around four times thicker than the overlying pre-treatment keratin (panel C). Without being bound by any theory or mode of action, a possible explanation for the increased thickness of the inner foreskin epithelium following treatment is an increase in epithelial cellular proliferation. This experiment demonstrates an increase in epithelial and keratin thickness of the inner foreskin epithelium following exposure to oestrogen.

For the initial experiments 0.5 ml of a topical oestriol cream (Ovestin-Registered Trade Mark of Organon, 1 mg/ml oestriol), applied once daily for 14 days was used. However, a lower dose may be used to achieve a clinically relevant outcome. For example, a two week daily application as described above followed by a once weekly maintenance dose of 500 μg may achieve such an outcome. The initial treatment period may also be equally effective using concentrations as low as 5 μg per day for 2 weeks. Optimally the final cream is formulated so that the recommended dose is contained within 100 μl of cream for ease of application. The final formulation could also be applied in an oil or aqueous solution from a dropper bottle or similar dispensing package.

Other oestrogenic compounds could also be used. Oestradiol has around 1000 times more potent systemic effects in vivo than oestriol and has an extended half-life. Oestrone is around 100 times more potent and has an intermediate half-life. While all metabolites appear to bind to the oestrogen receptor with the same affinity, oestradiol and oestrone have been shown to have more potent effects in vivo. The treatment proposed here is topical and only requires weak oestrogenic activity, therefore oestriol was chosen as the preferred oestrogenic compound.

Topical oestriol could be used in the range of 5 μg per application (per dose) to 500 μg per application (per dose); applied daily or weekly.

### REFERENCES

Short. (2006). New ways of preventing HIV infection: thinking simply, simply thinking. Phil. Trans. R. Soc. B. 361:811-820
Royce et al. (1997). Current concepts: sexual transmission of HIV. N. Engl. J. Med. 336: 1072-1078.
Schwartz. (2007). Langerhans cells lap up HIV-1. Nat. Med. 13(3): 245-246.
McCoombe and Short. (2006). Potential HIV-1 target cells in the human penis. AIDS 20: 1491-1495.
de Witte et al. (2007). Langerin is a natural barrier to HIV-1 transmission by langerhans cells. Nature Medicine 13 (3): 367-371.
Smith et al. (2004). Topical estrogen protects against Sly vaginal transmission without evidence of systemic effect. AIDS 18: 1637-1643.
Newell and Barninghausen. (2007). Male circumcision to cut HIV risk in the general population. Lancet. 369(9562):617-9.

1-13. (canceled)

14. A method of reducing or inhibiting the risk of infection from sexually transmitted viral infections (STIs) by increasing keratinisation of the human penile epithelium of a penis comprising topically administering a compound that induces keratinisation to a human penis.

15. The method of claim 14, comprising topically administering an oestrogenic compound, analogue or metabolite thereof, or an oestrogen agonist to a human penis.

16. The method of claim 14 in which the STIs are HIV and HPV.

17. The method of claim 16 in which the human penile epithelium is the inner foreskin of the penis.

18. The method of claim 17 in which the compound that induces keratinisation includes oestrogenic compounds, analogues or metabolites thereof, or oestrogen agonists.

19. A method of increasing keratinisation of a human penile epithelium of a penis, especially the inner foreskin of the penis, by topical administration to the penis of a compound.

20. The method of claim 19 in which the compound is an oestrogenic compound, analogue or metabolite thereof, or an oestrogen agonist.

21. The method of claim 19 in which the compound is a non-oestrogenic compound.

22. A method of decreasing the exposure of Langerhans cells in a human penis to sexually transmitted infectious agents by topical administration of an oestrogenic compound, analogue or metabolite thereof, or oestrogen agonist to the penis.

23. A topical oestrogenic compound, analogue or metabolite thereof or oestrogen agonist formulation when used in the methods of claim 14 to reduce or inhibit the risk of infection from sexually transmitted infections (STIs), such as HIV and HPV.

24. The topical oestrogenic compound, analogue or metabolite thereof or oestrogen agonist formulation of claim 23 adapted for external topical application.

25. The topical oestrogenic compound, analogue or metabolite thereof or oestrogen agonist formulation of claim 23 for use as a short-term pre- and post-operative medication for individuals about to undergo surgical circumcision.

26. A condom having a coating on at least part of its surface containing a formulation according to claim 23.

27. The method of claim 15 in which the STIs are HIV and HPV.

28. The method of claim 15 in which the human penile epithelium is the inner foreskin of the penis.
